(19) United States
(12) Patent Application Publication
(10) Pub. No.: US 2026/0341482 A1
(43) Pub. Date: Oct. 29, 2026

(54) RADIOLOGY WORKFLOW COORDINATION

(71) Applicant: KONINKLIJKE PHILIPS N.V., EINDHOVEN (NL)

(72) Inventors: THOMAS ERIK AMTHOR, HAMBURG (DE); XINYU WANG, HAMBURG (DE); THOMAS ANTEN, EINDHOVEN (NL); SIVA CHAITANYA CHADUVULA, MALDEN, MA (US); TANJA GERDES-NORDHOFF, HAMBURG (DE); JOACHIM DIETER SCHMIDT, HAMBURG (DE); RANJITH NAVEEN TELLIS, TEWKSBURY, MA (US); SANDRA VOSBERGEN, LIEMPDE (NL)

(21) Appl. No.: 19/121,454

(22) PCT Filed: Oct. 23, 2023
(86) PCT No.: PCT/EP2023/078121

§ 371 Date: Apr. 16, 2025

Related U.S. Application Data
(60) Provisional application No. 63/418,072, filed on Oct. 21, 2022.

(51) Int. Cl. G16H 40/20; G16H 30/20; G16H 80/00

(52) U.S. Cl. CPC G16H 40/20; G16H 30/20; G16H 80/00

(57) ABSTRACT

A radiology workflow assistance method includes retrieving information about an upcoming radiology examination of a patient, including information identifying an imaging modality of the upcoming radiology examination, information about the patient, and information obtained from a radiology examination order. Based on the retrieved information, role-specific examination complexity metrics are determined for respective personnel roles of a workflow for performing the upcoming radiology examination. Advice and/or assistance are provided to personnel assigned to fill the personnel roles of the workflow based on the determined role-specific examination complexity metrics. Assistance may also include producing a draft communication request by filling in a communication request form based on an inquiry and radiology examination context, enabling the requestor to edit and approve the draft communication request, and sending the approved communication request to a recipient identified therein.

### FIELD

The following relates generally to the radiology arts, medical imaging arts, radiology information technology (IT) support arts, and related arts.

### BACKGROUND

Radiology, also called medical imaging, is employed in a wide range of medical fields for diagnosis and monitoring of diseases and other medical conditions. Examples of radiological imaging modalities include magnetic resonance imaging (MRI), computed tomography (CT) and other X-ray imaging modalities, emission imaging modalities such as positron emission tomography (PET) and single photon emission computed tomography (SPECT), and so forth. Radiological imaging can also be classified by medical field, e.g. oncology imaging, sports medicine imaging, cardiac imaging, image guided therapy (iGT), and so forth. The radiology workflow is generally divided into several phases, including the image acquisition, image interpretation, and clinical application phases. Image acquisition is typically performed by an imaging technician or technologist or operator or the like. Image interpretation is typically performed by a radiologist, who is typically a medical doctor (e.g., an M.D.) specializing in interpretation of radiology image studies. Clinical application is typically performed by the patient's doctor, e.g. a general practitioner doctor or specialist such as an oncologist, cardiologist, orthopedic surgeon, or the like. The radiology workflow usually involves additional personnel participating in various additional roles, such as nurses, patient transport personnel, cleaning staff who clean and sanitize the medical imaging device and ancillary equipment between imaging examinations, clerical staff who maintain the radiology examination schedule, information technology (IT) staff who maintain the Picture Archiving and Communication System (PACS), Radiology Information System (RIS), and/or other IT infrastructure, a biomedical engineer (biomed) or other on-site engineer with qualification to perform some types of medical imaging device maintenance, a multilingual interpreter to provide translation for patients who do not speak the local language, and so forth.

Some radiology workflows also include additional personnel participating in remotely situated roles. For example, a Radiology Operations Command Centre (ROCC) may provide assistance to an imaging technologist and/or other personnel participating in a radiology examination. The ROCC provides a staff of remote experts performing the role of providing remote assistance to the imaging technologist performing a radiology examination via telephone, videocall, text messaging, or the like. The ROCC staff typically includes senior imaging technologists and/or radiologists who can provide additional expertise to assist less experienced local imaging technologists. Such assistance can be beneficial since the local imaging technologist may be expected to perform radiology examinations using a range of imaging modalities (MRI, CT, PET, et cetera) possibly manufactured by different vendors in support of a range of medical fields (oncology, cardiology, sports medicine et cetera), working with patients who may have a range of acute and chronic medical conditions. As another example, some medical institutions may outsource the radiology examination interpretation phase to a teleradiology department that employs a staff of radiologists on-call to perform remote radiology examination readings. As yet another example, the medical imaging device manufacturer or a third-party service provider may be called in to handle more complex medical imaging device maintenance issues that cannot be handled in-house by the on-staff biomed. Use of such classes of remote experts beneficially expands the capabilities of medical institutions without the concomitant human resources cost of employing experts specializing in these diverse areas.

Execution of a radiology examination thus involves cooperation amongst personnel serving in various roles. In one nonlimiting illustrative example, the patient's doctor writes a radiology examination order specifying a reason for examination and other relevant information. The order is received by clerical personnel of the radiology department who schedule the patient's examination. At the appointed time, the patient is transported to the radiology department (either on his or her own, or by transportation staff and/or a nurse depending on the patient's mobility), and the nurse, transportation staff, and/or the imaging technologist load the patient into the medical imaging device. The imaging technologist then sets up and executes imaging scans to acquire the medical images requested by the examination order. The images are reviewed by the imaging technologist (and possibly also by an on-call radiologist) to ensure they are of clinical quality, rescans may be done as appropriate, and the final clinical images and associated metadata are stored in DICOM format in the PACS. The patient is discharged, with the assistance of a nurse and/or transportation staff as needed based on patient mobility. At some time thereafter, a radiologist logs onto the PACS, downloads the images and associated metadata of the radiology examination, and performs a radiology reading of the images in light of the patient's electronic medical record and/or other available information, and memorializes the conclusions of this interpretation in a radiology report which is stored in the PACS and forwarded to the referring physician for clinical application. This is merely an example workflow.

A problem that can arise is that it can be difficult to efficiently coordinate the efforts of personnel in different roles to efficiently perform radiology examinations and maintain high throughput for the radiology department. Inefficiency or lack of communication can introduce delays in the workflow, or can even require rescheduling of radiology examinations at high cost to the radiology department, inconvenience to the patient, and potential detriment to patient care quality. As one illustrative example, if the imaging technologist determines at some point over the course of a radiology examination that he or she requires assistance from the ROCC, the imaging technologist then places a call to the ROCC. As this is an unscheduled request for assistance, there may be some delay before a remote expert at the ROCC is available to handle this assistance request. During which delay the patient may be stuck in the imaging scanner, and the delay can result in unanticipated backlog for the radiology examinations schedule. Similar delays can result from other unexpected events, such as a patient that presents special transport difficulties, a patient with an infectious disease such as COVID-19 requiring extra sanitation after the examination, and so forth.

Further difficulties can arise from poorly prepared communication requests. For example, if a request for assistance from the ROCC does not include certain important information, this can delay the assistance or can result in the request being handled by an ROCC staff member who is not sufficiently knowledgeable about the problem at hand to provide effective assistance. Conversely, if the requestor prepares a detailed request with a lot of information, this can in and of itself introduce delay as the requestor must locate the information which may be stored in various databases. Even with such effort, the requestor may fail to provide important information since the requestor is (by virtue of making the request) unfamiliar with how to solve the problem.

The following discloses certain improvements.

### SUMMARY

In one disclosed aspect, a radiology workflow assistance apparatus comprises an electronic processor and a non-transitory storage medium that stores instructions readable and executable by the electronic processor to perform a radiology workflow assistance method. That method includes: retrieving information pertaining to an upcoming radiology examination of a patient including at least information identifying an imaging modality to be used in the upcoming radiology examination, information about the patient, and information obtained from a radiology examination order for the upcoming radiology examination; based on the retrieved information, determining role-specific examination complexity metrics for respective personnel roles of a workflow for performing the upcoming radiology examination; and providing advice and/or assistance to personnel assigned to fill the personnel roles of the workflow based on the determined role-specific examination complexity metrics.

In another disclosed aspect, a radiology workflow assistance apparatus comprises an electronic processor and a non-transitory storage medium that stores instructions readable and executable by the electronic processor to perform a radiology workflow assistance method. That method includes: receiving an inquiry regarding a radiology examination via a requestor's electronic device operated by a requestor; producing a draft communication request by filling in fields of a communication request form based on the inquiry and a context of the radiology examination; providing a user interface on the requestor's electronic device which displays the draft communication request, enables the requestor to edit the draft communication request, and approve the draft communication request whereby the draft communication request becomes an approved communication request; and sending the approved communication request to a recipient identified in the approved communication request.

One advantage resides in providing advance notice to personnel participating in a radiology workflow of radiology examinations which may present a specific difficulty.

Another advantage resides in such advance notice tailored to specific roles which are expected to be impacted by the specific difficulty.

Another advantage resides in providing automated booking of a request for assistance to handle the specific difficulty.

Another advantage resides in providing role-specific predictions of complexities of upcoming radiology examinations, thereby enabling personnel participating in radiology examinations in the various roles to make advance preparation for upcoming radiology examinations complexities.

Another advantage resides in providing automated or semi-automated construction of an assistance request with sufficient information to facilitate resolution of the request.

Another advantage resides in providing such automated or semi-automated construction of an assistance request based in part on context of the radiology examination to which the assistance request pertains.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a radiology workflow assistance apparatus in the context of a diagrammatically represented radiology examination environment

FIG. 2 diagrammatically illustrates an embodiment of the role-specific examination complexity metric prediction of FIG. 1.

FIG. 3 diagrammatically illustrates a radiology schedule-based user interface (UI) for a remote imaging technologist.

FIG. 4 diagrammatically illustrates a radiology schedule-based UI for an on-site imaging technologist.

FIG. 5 diagrammatically shows an example of a role-specific complexity metric in the form of a multidimensional vector.

FIG. 6 diagrammatically shows an illustrative embodiment of the process-aware assistance request process of FIG. 1.

FIG. 7 diagrammatically illustrates a graphical user interface (GUI) representation of a radiology examination workflow timeline.

### DETAILED DESCRIPTION

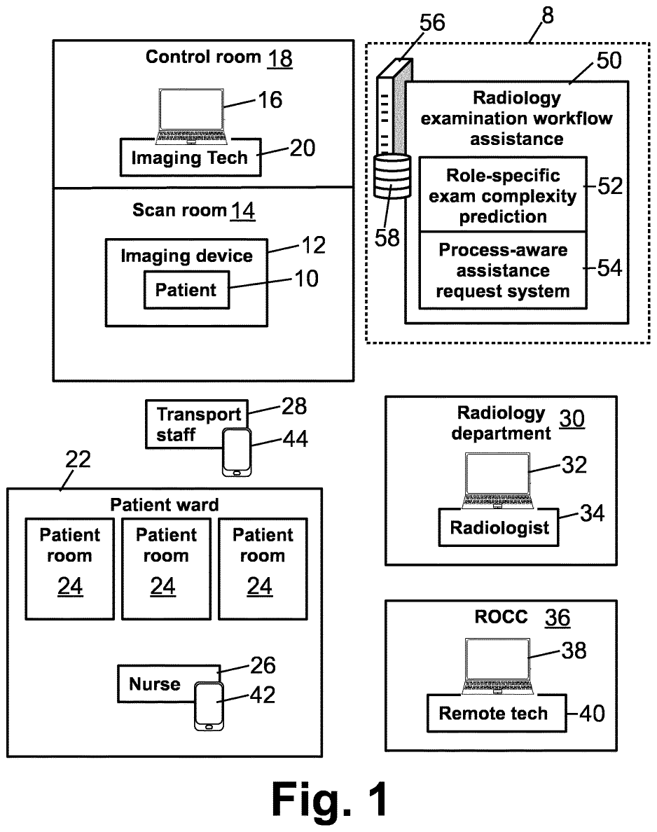

FIG. 1 diagrammatically illustrates a radiology workflow assistance apparatus 8 in the context of a diagrammatically represented radiology examination environment. The radiology examination is performed on a patient 10 using a medical imaging device 12. As used herein, the term “patient” merely indicates an individual undergoing a radiology examination—the patient 10 may be a hospital patient, an outpatient, or a person undergoing a routine screening radiology procedure. In a typical arrangement, the imaging device 12 is located in a scan room 14, and an imaging device controller 16 operatively connected to control the imaging device 12 is located in an adjacent control room 18 controls the medical imaging device 12, usually separated by a wall with a large window so that an imaging technologist 20 can observe the scan room 14 where the patient is located while the imaging technologist 20 is in the control room 18. The use of the illustrative separate control room 18 is common in the case of imaging modalities such as MRI or CT which can produce strong magnetic/electromagnetic fields, ionizing radiation or so forth, but may be omitted in the case of some imaging modalities or in some specific radiology department layouts, in which case the imaging device 12 and imaging device controller 16 are suitably located in a single room. The medical imaging device 12 may in general be of any type or imaging modality used for imaging a human body for medical diagnostic, monitoring, screening, or other like purposes. The medical imaging device 12 may by way of nonlimiting illustrative example comprise an MRI scanner, CT scanner or other X-ray imaging modality, PET scanner, SPECT scanner, a multimodality imaging device such as a CT/PET scanner, or so forth. While the imaging technologist 20 is shown in FIG. 1 located in the control room 18, the imaging technologist 20 will typically move back and forth between the scan room 14 and the control room 18, e.g. going into the scan room 14 to prepare and load the patient 10 into the medical imaging device 12, back into the control room 18 to set up and start scanning using the imaging device controller 16, and return to the scan room 14 as appropriate to perform actions such as positioning a local MRI coil, and eventually to unload and prepare the patient for discharge. The personnel role filled by the imaging technologist 20 may be otherwise named, e.g. as an imaging technician, radiology technologist, imaging device operator, or so forth.

With continuing reference to FIG. 1, the diagrammatically represented radiology examination environment is shown as an extended environment which includes locations of additional personnel participating in the radiology examination. By way of nonlimiting illustrative example, a patient ward 22 is shown, with a number of patient rooms 24. In cases in which the illustrative patient 10 is a hospital patient (i.e., an in-patient), the patient may ordinarily reside (i.e., while admitted to the hospital) in one of the patient rooms 24 assigned to the illustrative patient 10, and may be cared for nurses represented by an illustrative nurse 26. The radiology examination workflow then begins by the nurse 26 and/or a member of the hospital's transportation staff 28 preparing and transporting the patient 10 from the patient ward 22 to the scan room 14 and may also perform or assist in performing the preparation and loading of the patient 10 into the imaging device 12. (On the other hand, if the patient 10 is an outpatient or a screening patient, the patient 10 may be sufficiently mobile to transport himself or herself to the radiology department without such assistance, although the imaging technologist 20, nurse 26, and or transport staff 28 may still assist in preparing and loading the patient 10 into the imaging device 12. Other personnel roles may participate in the coordination of patient arrival and discharge who are not shown, such as a receptionist or other clerical staff who may handle adding the radiology examination to the radiology examination schedule, a translator if needed, and/or so forth.

With continuing reference to FIG. 1, yet other personnel roles may be involved in the radiology examination workflow. As nonlimiting illustrative examples, FIG. 1 further depicts a radiology department 30 containing a radiology workstation 32 where a radiologist 34 is on staff during the radiology examination, and a remote operations control center (ROCC) 36 containing a remote imaging technologist workstation 38 where a remote (and typically senior) imaging technologist 40 is on call to provide remote assistance to the local imaging technologist 20. The personnel role filled by the remote imaging technologist 40 may be otherwise named, e.g. as a remote imaging technologist, remote radiology technologist, remote expert, or so forth.

In a typical radiology examination workflow, the workflow is initiated by a radiology examination order issued by a referring physician (not shown), such as the general practitioner (GP) physician of the patient 10, or a specialist such as an oncologist, cardiologist, or so forth. The amount of detail in the radiology examination order may depend on how knowledgeable the referring physician is about radiology, but in general the radiology examination order will usually contain at least a reason for examination, the imaging modality to be used, and an identification of the patient 10 (e.g. by patient name, social security number, patient identification number used in the hospital, various combinations thereof, and/or so forth). The on-call radiologist 34 (or possibly some other radiologist) may prepare a more detailed imaging protocol or instructions on the basis of the radiology examination order. During the radiology examination, the on-call radiologist 34 may provide consultative assistance to the local imaging technologist 20 in deciding details about imaging scan or protocol configuration, advice on handling unexpected challenges (e.g., an obese patient, a pediatric patient who is unable to stay still during imaging data acquisition, or so forth). The on-call radiologist 34 may also, in some radiology examination workflows, be expected to provide an initial review of the acquired images to ensure they are of sufficient quality, type, and field of view to provide the information needed to fulfill the radiology examination order. (Such a “quality control” review by the on-call radiologist 34 would precede the actual clinical reading of the radiology examination during which the illustrative radiologist 34 or some other radiologist reviews the images in detail and writes up a radiology report presenting the radiologist's findings). To provide such review, the on-call radiologist 34 may physically walk into the control room 18 to review the images on the display of the imaging device controller 16, or those images may be transferred via an electronic network to the radiology workstation 32 for review by the on-call radiologist 34 there stationed.

The ROCC 36 is also an optional component of the radiology examination workflow. If provided, the remote imaging technologist 40 staffing the ROCC 36 is on call to assist local imaging technologists during radiology examinations, including being on call to assist the illustrative local imaging technologist 20 performing the radiology examination on the illustrative patient 10. The remote imaging technologist 40 is typically an experienced and/or senior imaging technologist who can provide assistance to the (possibly less experienced and/or more junior) local imaging technologist 20 actually performing the radiology examination. Such assistance may be provided by telephone, videocall, text messaging, or the like. To provide the remote imaging technologist 40 with situational awareness so he or she can provide useful assistance, one or more cameras, microphones, and/or other sensors (not shown) may be located in the scan room 14 and/or control room 18 with the feeds from these cameras, microphones, or other sensors being delivered to and presented by the remote imaging technologist workstation 38. Additionally or alternatively, a video splitter, screen-sharing software, or the like can be provided to capture the display of the imaging device controller 16 in real-time and present it on the remote imaging technologist workstation 38 for viewing by the remote imaging technologist 40.

As further shown in FIG. 1, each of the illustrative persons filling personnel roles of, and participating in, the radiology workflow typically have an electronic device. By way of nonlimiting illustrative example, the local imaging technologist 20 has the imaging device controller 16 and may also have one or more other electronic devices such as a tablet computer for referring to the radiology schedule, providing videocalls with the on-call radiologist 34 and/or the remote imaging technologist 40, and/or so forth. The radiologist 34 is shown with his or her electronic device comprising the illustrative radiology workstation 32. The remote imaging technologist 38 is shown with his or her electronic device comprising the illustrative remote imaging technologist workstation 38. In the case of nurses 26 and transportation staff 28 who are typically more mobile, the corresponding electronic device may for example be an illustrative nurse's cellular telephone (cellphone) 42 and an illustrative transport staff person's cellphone 44, respectively. More generally, a specific person's electronic device is an electronic device that is accessible at least by that specific person. As some nonlimiting illustrative examples, a specific person's electronic device may be a cellphone, tablet computer, workstation, or the like to which the specific person is logged in, or an imaging device controller, radiology department computer, or the like which is generally accessible to persons including the specific person, or which is accessible by persons in a personnel role filled by the specific person. In some such cases, the specific person could correspond to a group of people interchangeably filling a personnel role of the radiology examination workflow, e.g. in the case of the nurse 26 his or her electronic device could additionally or alternatively be an electronic whiteboard of a nurses' station of the patient's ward 22. These are merely nonlimiting illustrative examples. In any of these cases, content may be presented on the specific person's electronic device. Presenting content on a person's electronic device which is accessible by the person refers to causing the person's electronic device to present the content. Typically, the presentation will be by displaying the content on an LCD display, LED display, OLED display, or other display of the person's electronic device. However, the content could be presented on the person's electronic device in other human-perceptible ways, such as by way of a computer-generated voice output by a loudspeaker of the electronic device, or as virtual reality (VR) or augmented reality (AR) content in a case where the person's electronic device is a VR or AR headset. It may be noted that in some cases there could be time lag for the presentation. For example, if the person's electronic device is a cellphone then the content may be displayed only when the person logs into or unlocks the cellphone. In such cases, in some embodiments an audible notification tone may be issued to inform the person that the content is available.

To further illustrate a typical extended radiology examination environment, Table 1 provides some examples of processes in the radiology examination workflow along with the personnel roles involved, corresponding tasks, and information sources to obtain information about these tasks.

TABLE 1

| | | | | Possible |
|---|---|---|---|---|
| | | | | data |
| | Process | Role | Task | source*** |
| Before | Patient arrival | Receptionist/Nurse | Check- | RIS |
| exam | | | in/Registration | |
| starts | Language assistance | Interpreter | Facilitate | — |
| | | | communication | |
| | | | for patient | |
| | Patient education | Nurse/Technologist | Inform patient | — |
| | | | of procedure | |
| | Implant | Nurse/Technologist | If implant exists, | RIS |
| | compatibility | | check its | |
| | check* | | compatibility | |
| | Sedation* | Anesthesiologist | Sedate patient | RIS |
| | IV access* | Nurse/Technologist/ | Set up IV access | — |
| | | Phlebotomist | | |
| During an | Place patient** | Nurse/Technologist/ | Place patient on | RIS, |
| exam | | Transportation staff | scanner table | Camera |
| | Positioning & coil | Technologist | Set focus on | Device log |
| | placement | | anatomic region | data |
| | Contrast agent setup | Technologist/ | Setup contrast | |
| | | Radiologist | | |
| | Order/Protocol | Referring Physician | Order/Protocol | |
| | changes | | change approval | |
| | Protocol set up and | Technologist | Set up exam | Device log |
| | geometry planning | | protocol and | data |
| | | | plan geometry | |
| | Image acquisition | Technologist | Follow and | Device log |
| | | | monitor | data |
| | | | scanning | |
| | Image quality check | Technologist/ | Ensure image | — |
| | | Radiologist | quality | |
| After | Patient release** | Nurse/Transportation | Help patient to | Camera |
| exam | | staff/Technologist | move out of | |
| ends | | | scanner room | |
| | Reporting and paper | Technologist | Send image to | PACS, HL7 |
| | work | | PACS | |
| | Room | Cleaning staff | Clean and | Sensor |
| | cleaning/disinfection | | disinfect scanner | |
| | | | room | |

FIG. 1 and Table 1 provide nonlimiting illustrative examples of an extended radiology examination environment to illustrate that successful execution of the radiology examination entails participation, cooperation, and communication amongst clinicians and other persons serving in a wide range of personnel roles, such as the illustrative (local) imaging technologist 20, remote imaging technologist 40, on-call radiologist 34, nurse(s) 26, transportation staff 28, and/or potentially other personnel roles not shown such as clerical staff. The various participating persons' electronic devices 16, 32, 38, 42, 44 are typically connected to an electronic network (e.g., including the hospital IT network, the Internet, and/or so forth) enabling wired and/or wireless communication between the various persons participating in the radiology examination via email, text message, audio- or videocall using these devices. However, scheduling and/or initiating such communication amongst persons participating in the radiology examination workflow can be challenging. A request for assistance from a requestor can amount to an undesirable interruption for the requestee. A communication request due to an unanticipated problem can be particularly difficult to accommodate. Additionally, if the request sent by the requestor is incomplete, time can be wasted as the requestee collects the missing information. In some circumstances, a requestor may also be unsure of to whom the request should be directed.

The radiology workflow assistance apparatus 8 operating in the context of the radiology examination environment advantageously facilitates more effective communication amongst persons participating in the radiology examination workflow. As diagrammatically shown in FIG. 1, the illustrative radiology workflow assistance apparatus 8 implements various radiology examination workflow methods or sub-systems 50, such as a role-specific examination complexity metric prediction 52 and a process-aware assistance request system 54. The radiology workflow assistance apparatus 8 may be suitably embodied by an electronic processor 56 (for example, an illustrative server computer 56 or other computer with suitable IT network connectivity and processing power) and a non-transitory storage medium 58 storing instructions readable and executable by the electronic processor to implement the radiology examination workflow assistance 50. The non-transitory storage medium 58 may for example comprise a hard disk, solid state disk (SSD), flash memory, optical disk, various combinations thereof, and/or the like. It will be appreciated that the electronic processor 56 may be implemented as multiple intercommunicating electronic processors, and likewise the non-transitory storage medium 58 may be implemented as a plurality of non-transitory storage media.

The role-specific examination complexity metric prediction 52 provides advance estimation to persons participating in the radiology examination workflow as to how complex the radiology examination will likely be. This can reduce the problem of unanticipated requests for assistance by providing advance notice of radiology examinations that are likely to involve such requests. The role-specific examination complexity metric prediction 52 advantageously provides different complexity metric predictions for different personnel roles, since a given radiology examination may be more or less complex for persons filling different personnel roles. As just two examples, if the patient 10 is frail then this may greatly increase complexity for the nurse 26 and transport staff 28 as they may need a gurney, wheelchair or other transport apparatus, and/or may need additional persons to assist the patient. On the other hand, frailty of the patient 10 may present less of a challenge to the local imaging technologist 20, and may present almost no additional complexity for the on-call radiologist 34. On the other hand, if the patient 10 is undergoing a CT examination and has a metal implant in or near the anatomy to be imaged, this can greatly increase complexity of the radiology examination for the local imaging technologist 20, remote imaging technologist 40 (who may likely be called to provide advice or verbal assistance), and the on-call radiologist 34 who will need to carefully review the images to ensure the metal implant has not shadowed or occluded relevant anatomy. But the metal implant is likely to have little or no impact on examination complexity for the nurse 26 and transport 28.

In one approach, the role-specific examination complexity metric prediction 52 retrieves information pertaining to an upcoming radiology examination of the patient 10 including at least information identifying an imaging modality to be used in the upcoming radiology examination, information about the patient 10, and information obtained from a radiology examination order for the upcoming radiology examination; based on the retrieved information, determining role-specific examination complexity metrics for respective personnel roles of a workflow for performing the upcoming radiology examination; and providing advice and/or assistance to personnel assigned to fill the personnel roles of the workflow based on the determined role-specific examination complexity metrics. In some embodiments, the role-specific examination complexity metric comprises a multidimensional vector in which each dimension of the multidimensional vector contains a value indicative of role-specific complexity of the upcoming radiology examination with respect to a corresponding aspect of the upcoming radiology examination. This can enable further narrowing the timing and scope of a likely request for assistance.

The process-aware assistance request system 54 provides context-aware assistance in preparing a request for assistance. In one illustrative embodiment, in response to receiving an inquiry regarding a radiology examination via a requestor's electronic device operated by a requestor (e.g., one of the participating persons' electronic devices 16, 32, 38, 42, 44), the process aware assistance request system 54 produces a draft communication request by filling in fields of a communication request form based on the inquiry and a context of the radiology examination, and provides a user interface (UI) on the requestor's electronic device which displays the draft communication request, enables the requestor to edit the draft communication request, and approve the draft communication request whereby the draft communication request becomes an approved communication request. The assistance request system 54 then sends the approved communication request to a recipient identified in the approved communication request (for example, by sending it to another of the participating persons' electronic devices 16, 32, 38, 42, 44, this one associated with the recipient).

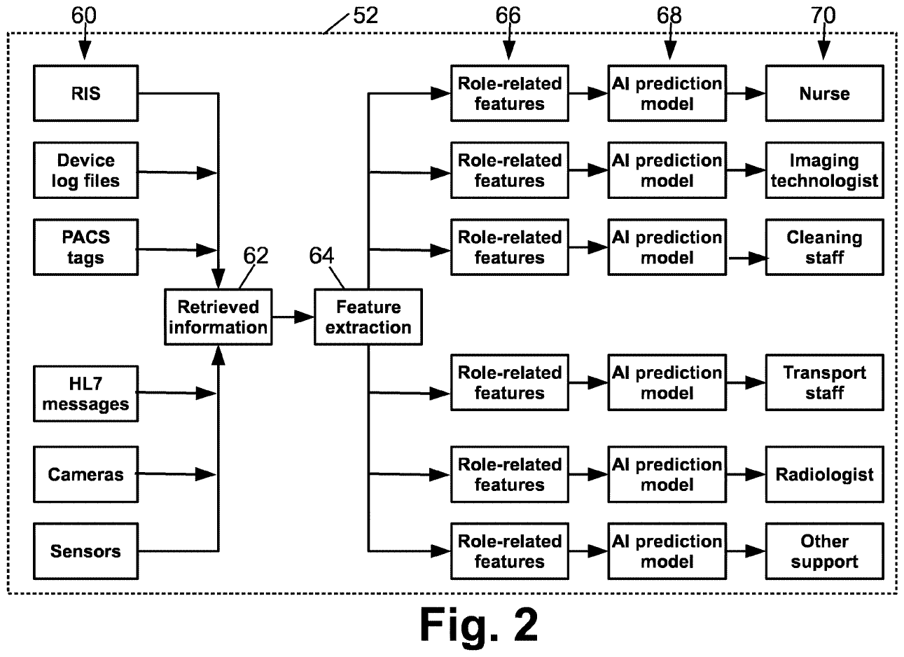

With reference to FIG. 2, an illustrative embodiment of the role-specific examination complexity metric prediction 52 is described. In a first step, information pertaining to an upcoming radiology examination of the patient 10 is obtained. Typically, this information includes at least information identifying an imaging modality to be used in the upcoming radiology examination, information about the patient, and information obtained from a radiology examination order for the upcoming radiology examination. The information may be obtained from a variety of information sources 60, such as: a radiology information system (RIS) which may for example store the radiology examination order (typically containing at least the patient identification and the imaging modality and the reason for examination) and the radiology schedule including information about scheduled patients and personnel assignments for various personnel roles in the radiology examination workflow (or scheduled for the radiology work shift of the upcoming radiology examination); medical imaging device log files; PACS tags; HL7 messages; cameras and sensors used by the ROCC 36 (if available), and/or so forth. The information sources 60 listed in FIG. 2 are to be understood as nonlimiting illustrative examples. Retrieval of information from these information sources 60 may be by way of a set of scripts that retrieve permitted and anonymized data from available data sources, parse, clean the data and store it in a local or remote protected database under predefined schemas.

An automated process 62 filters the retrieved data, and a further automated feature selection process 64 selects role-related features 66 for each personnel role based on the tasks of the targeted personnel roles (e.g., such as the roles listed in Table 1 and/or shown in FIG. 1). In one embodiment, a process model (such as a business process model) is used to define all activities (tasks) in the process and the respective human resources connected to each activity. In this way, the roles of staff involved in a particular activity can be identified. For example, for imaging technologists, their tasks are mainly to carry out scanning examinations. The complexity of their tasks depends on patient condition, exam characteristic, as well as their own level of expertise. Therefore, these features will be useful inputs to role-specific predictive models implemented, for example, as artificial intelligence (AI) models. On the other hand, for transportation staff, their tasks mostly involve transporting in-patients from hospital wards to the scanning bays. Patient condition is the most relevant factor for their tasks, and thus would be a valuable feature for the transport staff role-specific predictive model; whereas examination characteristics do not influence so much the need for transport but could be a proxy in the absence of unknown patient characteristics.

The output of the automated processes 62 and 64 is a set of role-related features 66 for each personnel role participating in the radiology examination workflow. For each personnel role, a corresponding role-specific predictive model 68 is applied to the role-related features 66 to generate the role-specific examination complexity metric 70 for that personnel role. In FIG. 2, the boxes representing the output role-specific examination complexity metrics 70 are labeled by the corresponding roles, e.g. “Nurse”, “Imaging technologist”, “Cleaning staff”, “Transport staff”, “Radiologist”, and “Other support” (for example, the remote imaging technologist 40 of FIG. 1). In one nonlimiting illustrative embodiment, the role-specific predictive models 68 are trained AI models such as artificial neural network (ANN) models. In embodiments in which the role-specific examination complexity metric 70 of a role comprises a multidimensional vector in which each dimension of the multidimensional vector contains a value indicative of role-specific complexity of the upcoming radiology examination with respect to a corresponding aspect of the upcoming radiology examination, a supervised model that supports multiclass output can be used. Depending on the role-related features set 66, the model could vary. For example, if the feature set selected for imaging technologists is mixed with categorical and numeric data, a random forest classifier may be a suitable choice. If the feature set selected for another role is composed of merely numeric data, then a support vector machine (SVM) classifier may be a suitable choice. These are merely nonlimiting illustrative examples. To provide role-specific examination complexity predictions, the applied predictive models 68 can vary for each targeted role, determined by the characteristic of the selected feature set. In another embodiment, several statistical and neural network algorithms can be tested to determine the one with best performance on the test data and the one that generalizes well on unseen data.

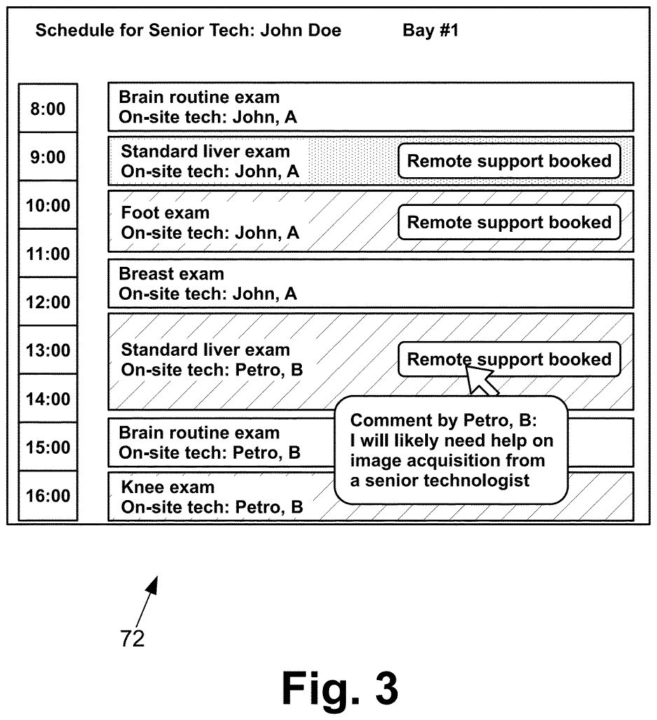

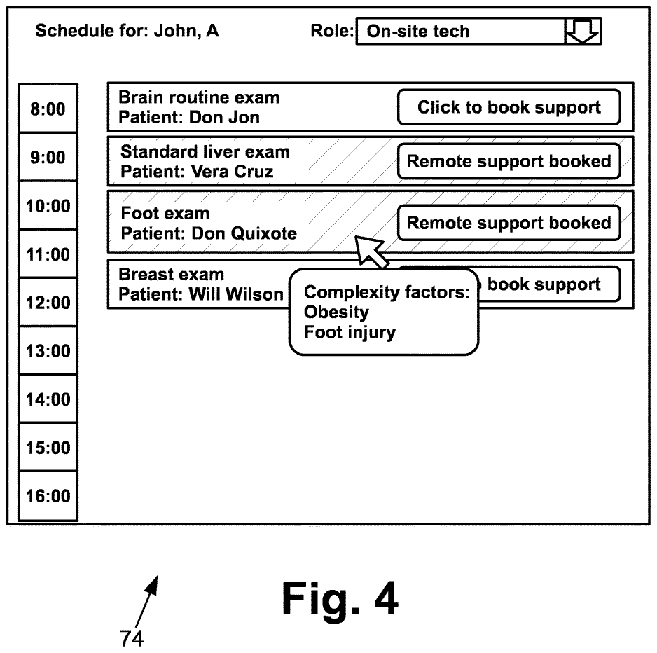

With reference to FIGS. 3 and 4, the output role-specific examination complexity metrics 70 from FIG. 2 can be used to provide advice and/or assistance to personnel assigned to fill the personnel roles of the workflow, for example via a radiology schedule-based user interface (UI) as shown in FIGS. 3 and 4. The radiology schedule-based user interface can be realized, for example, by web- or app-based application program interfaces (APIs). FIG. 3 illustrates such a radiology schedule-based UI 72 for a senior (i.e. remote) imaging technologist 40, while FIG. 4 illustrates such a radiology schedule-based UI 74 for an on-site (i.e. local) imaging technologist 20. Each scheduled upcoming radiology examination is listed on the schedule as a row with the scheduled time block to the left. Each scheduled upcoming radiology examination is color-coded in accordance with the senior technologist-specific complexity metric 70 computed by the process of FIG. 2, e.g. red may be used for a high-complexity prediction, yellow for an intermediate-complexity prediction, and green (or white, i.e. uncolored) for a low-complexity prediction. (In FIGS. 3 and 4, the color coding is diagrammatically shown by different types of shading or hatching-optionally, such shading or hatching and/or other types of highlighting could actually be substituted for color coding). Notably, since the UI 72 of FIG. 3 is for a senior ROCC imaging technologist while the UI 72 of FIG. 4 is for the local (on-site) imaging technologist, the role-specific complexity metrics 70 could be different. This is illustrated in FIGS. 3 and 4 in that the 9:00 upcoming radiology examination (standard liver examination of patient Vera Cruz) have different shading in FIGS. 3 and 4, reflecting different complexity metrics determined for the senior ROCC imaging technologist as compared with the local (on-site) imaging technologist. Another difference is that the radiology examinations from 12:30 and later are listed on the UI 72 of the senior technologist but are omitted from the UI 74 of the local technologist, reflecting the fact that (in this example) the local technologist is going off-duty at 12:30. While FIGS. 3 and 4 provide two illustrative UI for two different personnel roles, more generally, a different role-specific schedule-based UI may be provided for each personnel role, and the appropriate UI is presented on the respective participating persons' electronic devices 16, 32, 38, 42, 44 depending on their role.

The UI may also include other features implemented using suitable graphical user interface (GUI) techniques and user interfacing (e.g. via a mouse pointer or touch screen for example). In some embodiments, by hovering over the name of each technologist, it is possible to view the title and basic professional information of this technologist. Furthermore, by hovering over an exam, one can view reasons for the assigned role-specific complexity metric (as indicated by the color coding), e.g. why this exam is marked as ‘less likely requiring support’, ‘likely requiring support’ or ‘very likely requiring support’, or so forth. An example of this is shown in FIG. 4 as the pop-up bubble for the examination at 10:00 (Foot exam for patient Don Quixote). This is a summarized text which can be automatically composed by the most contributing features of the role-related features 66, which may be suitably output by the AI prediction models 68. Moreover, it is optionally possible to view which technologist has booked/requested support for which scheduled exam. When hovering over the yellow button, one can see the message/comment left by on-site technologist, highlighting what support is expected, as shown for the 13:00 radiology examination in FIG. 3. The UI 72 for the senior technologist (who may be scheduled to simultaneously support two or more concurrent radiology examinations) can optionally highlight any scheduling conflicts that require simultaneous support. The example UI 74 for the local technologist provides buttons indicating “remote support booked” (from the ROCC) if this is the case, or “Click to book support” if support has not yet been booked. These selection buttons suitably call up an ROCC support scheduling UI (not shown) that enables the local technologist to book support, including adding a comment on what assistance is being requested which is made available to the senior technologist, for example by the pop-up window as shown in FIG. 3. is. More generally, the UI for different personnel roles may provide various information and/or control options appropriate to specific roles.

While different UI are provided for different roles, optionally the UI can be switchable between roles, for example using the Role: drop-down list user dialog shown in the upper right of the UI 74 of FIG. 4.

With reference back to FIG. 2, in embodiments in which the role-specific predictive models 68 are AI models, various approaches can be used for training, utilizing various training data sources. For training AI prediction model, a set of features and known complexity values (“ground truth”) are usually supplied. Once trained, the AI prediction model is able to predict a complexity value when provided with the features. The ground truth can be collected in different ways, such as: by means of questionnaires or electronic feedback collection via a graphical user interface; by a staff member asked to rate the complexity of a task or activity just performed; indirectly by measuring the deviation of certain workflow parameters from expected average values (for example, an exam taking much longer than a typical exam of the same kind may be rated more complex); by measuring whether a request for support related to a certain activity has taken place, for example by monitoring the support requests or support calls in a ROCC setup (activities that are associated with support requests are then rated more complex than others); and so forth. Features provided to the AI prediction algorithm may include, by way of nonlimiting illustrative example: exam details (requested protocol, clinical question, referral information, . . . ); patient details (inpatient/outpatient, age, weight, gender, medical history, medication, special requirements, mobility, communication ability, . . . ); operation context (time of day, day of week, day of year, room, modality identifier, . . . ); profile/capabilities of staff member/role assigned to an activity (experience, specialties, language capabilities, . . . ); and so forth.

In some embodiments, the role-specific predictive models 68 output a single complexity metric, which may optionally have low granularity (e.g. low, medium, or high complexity). In other embodiments, instead of a single-valued complexity score (such as a scale of low, medium, high complexity), the complexity score may be represented as a multidimensional vector in which each element (i.e. dimension) of the complexity vector represents one aspect of the complexity.

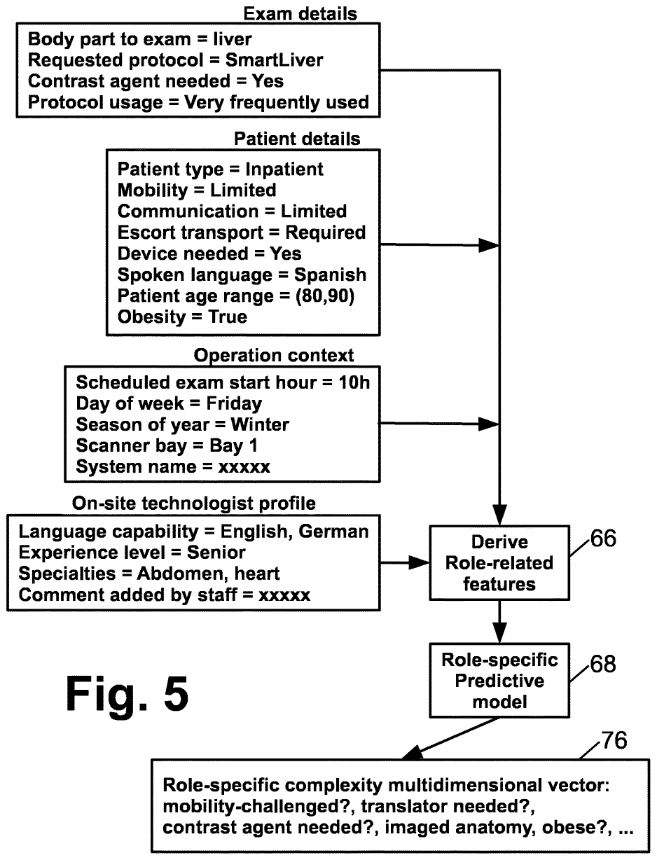

With reference to FIG. 5, an example of such a role-specific complexity metric in the form of a multidimensional vector 76 is diagrammatically shown. In this nonlimiting illustrative example, input features for computing the multidimensional vector 76 may, by way of nonlimiting illustrative example, include: mobility challenged?; translator needed?; contrast agent needed?; imaged anatomy; obese?; . . . . The multidimensional vector 76 contains predicted complexity scores for several aspects. The score value may, for example, be between 0 and 1 so as to map to likelihood. For example, a score of 0.1 means ‘support is unlikely needed’, whereas a score of 0.9 means ‘support is very likely needed’. An example of output could be:
“Need for physical assistance”=0.9;
“Need for language support”=0.9;
“Support for geometry planning”=0.1;
   The input features fed into the predictive model 68 might be that, patient is mobility-challenged, or patient is obese. Therefore, the predictive model 68 assigns a high score of 0.9 to aspect “Need for physical assistance”. The multi-dimensional complexity score (i.e. vector 76) expresses all aspects of the complexity at the same time. All aspects of the complexity can optionally be visualized to the workflow participant separately, because separate actions may be required to mitigate the expected difficulties. For example, when hovering the mouse pointer over the exam in the schedule UI 72 or 74 the elements of the vector may be shown in a pop-up box (not shown). Training the AI model for multi-dimensional complexity scores utilizes ground truth for all complexity aspects. Different ways of acquiring the required feedback as previously discussed may be used simultaneously in this case, to make sure that all aspects of the complexity are covered.

In general, the role-specific examination complexity prediction 52 may provide various types of advice and/or assistance to personnel assigned to fill the personnel roles of the radiology examination workflow based on the determined role-specific examination complexity metrics. FIGS. 3 and 4 present some examples, such as color-coding the schedule based on the complexity metrics, or more generally presenting information indicative of the role-specific examination complexity metrics 70 on electronic devices 16, 32, 38, 42, 44 accessible by the personnel 20, 26, 28, 34, 40 assigned to fill the personnel roles of the radiology examination workflow. Since the complexity metrics are role-specific, this entails presenting information indicative of each role-specific examination complexity metric 70 on one or more electronic devices 16, 32, 38, 42, 44 accessible by one or more persons 20, 26, 28, 34, 40 assigned to fill the personnel role of the workflow corresponding to that role-specific examination complexity metric. As previously noted, such presentation could be delayed, for example until the user unlocks his or her cellphone, and perhaps until the user opens the appropriate application program (app). Optionally, the app may push a notification, e.g. an audible beep and/or a text message showing on the lock screen of the cellphone, so that the user is immediately made aware that the presentation is available. Such a notification may be configurable, e.g. the user can use the “settings” of the cellphone to turn off or modify notifications.

In another example of advice and/or assistance, the role-specific examination complexity prediction 52 may predict assistance to a first personnel role of the workflow will be provided by a second personnel role of the workflow, and automatically schedule an electronic call (e.g. telephonic or videocall) between one or more persons filling the first personnel role of the workflow and one or more persons filling the second personnel role of the workflow. For example, this could include predicting a time at which the assistance to the first personnel role of the workflow is predicted to be provided by the second personnel role of the workflow, and the automatic scheduling includes scheduling the electronic call for the predicted time. In some embodiments, an expected duration of the assistance may also be predicted. In some such embodiments in which an electronic call is automatically scheduled, the time at which the assistance is predicted to be provided and the predicted duration of assistance may be used to set the time block of the scheduled call. As one nonlimiting specific example, the first personnel role of the workflow could be the local imaging technologist 20 operating the medical imaging device 12 in the workflow and the second personnel role of the workflow could be the remote imaging technologist 40 who does not operate the medical imaging device in the workflow. As another nonlimiting specific example, the first personnel role of the workflow could be the local imaging technologist 20 operating the medical imaging device 12 in the workflow and the second personnel role of the workflow could be the radiologist 34 scheduled to approve images acquired by the local imaging technologist 20 in the workflow.

It will be appreciated that the role-based complexity scored provided by embodiments such as the illustrative embodiments of FIGS. 2-5 beneficially provide personnel with role-based information for anticipating whether they may need to make a communication request. The system also provides recipients of such requests with advance notice of likely communication requests.

With reference back to FIG. 1, in the following, some illustrative embodiments of the process-aware assistance request system 54 is described. The system may suitably include: a workflow management system providing real-time process context; a user interface for request initiation and pre-selection of a subset of request items based on the choice of UI element that was selected for triggering the request, adaptation of proposed items and providing additional context where needed, and approval; a machine learning model to predict probabilities for any remaining request items; and a user interface for selection of proposed remaining request items. In some embodiments, a model of the radiologic processes is provided, in which the status of the running process instances is known at any time. The assistance request system 54 also employs a suitable pathway for processing and transmitting communication requests, such as via telephonic, videocall, or text messaging pathways for example. The assistance request system 54 may utilize APIs for interoperating with commercially available videoconferencing systems, with the APIs ensuring conformance to data privacy, data security, and medical device regulations as may be appropriate for a given implementation.

A communication request can be, for example, a message sent to another participant in the radiologic workflow, including a specific question and information about the context; or a ticket submitted to a ticketing system, to be picked up and processed by another participant in the radiologic workflow. In one suitable implementation, a request is defined as a set of request items, for example entered by filling in fields of a communication request form. The fields may include, by way of nonlimiting illustrative example: an identification of the intended recipient of the request; an identification of the patient whom the request is about; information about the type of the activity in the radiologic process to which the request is connected (e.g., patient preparation, imaging examination, etc.); a reference to the point in time when the content of the request is or will become relevant (e.g., the time when the activity is scheduled or predicted to happen); the type of request (type of question or requested action, e.g. “image quality check” or “protocol modification approval”); a description of the request details in addition to the type of request; an indication on urgency; information about the state of the current process within the department; and/or so forth. A given request may include only a subset of these fields, and/or may include additional fields.

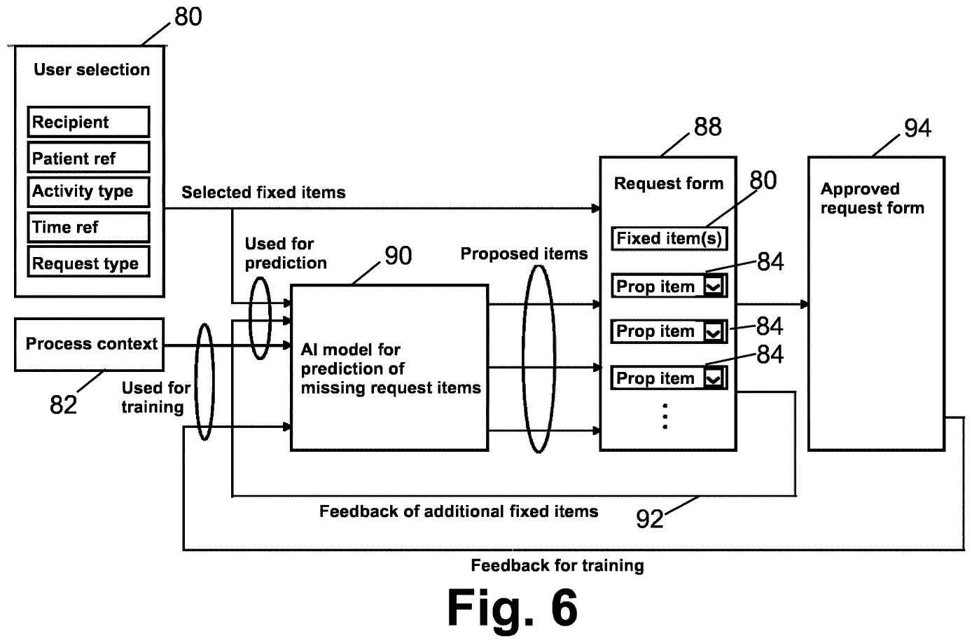

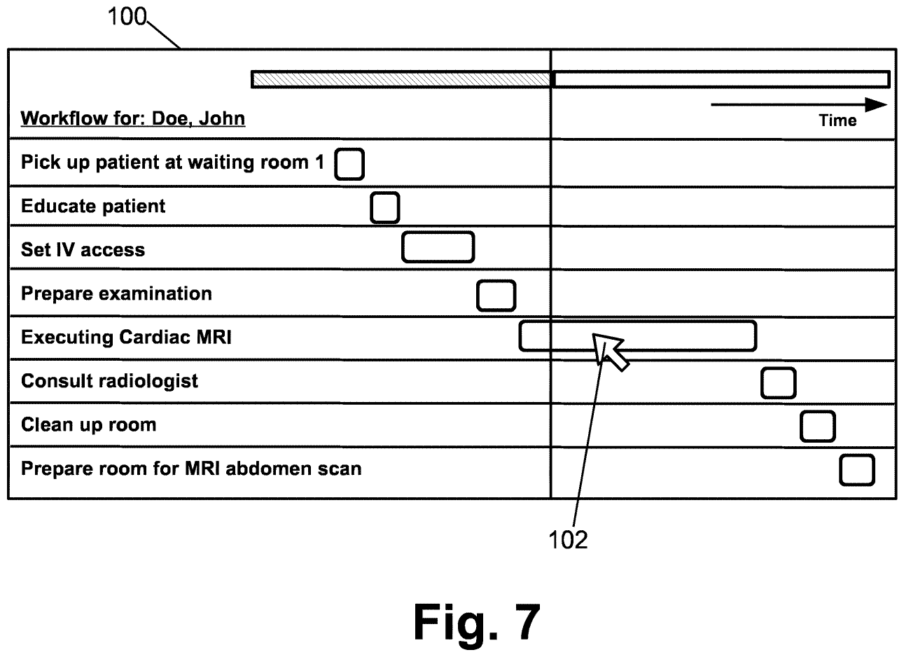

With reference to FIG. 6, an illustrative embodiment of a process-aware assistance request process performed by the system 54 is shown. In a first step, a user initiates a communication request by way of submitting an inquiry regarding a radiology examination via a requestor's electronic device operated by a requestor. The inquiry provides a subset 80 of the fields (also referred to herein as request items) of a communication request form. In one suitable approach, the inquiry is initiated by the user selecting a corresponding element in a graphical representation of the radiology examination workflow (see FIG. 7 for an example). In this way, the requestor implicitly selects some request information simply by initiating the request by clicking a relevant item on the screen. More generally, the system 54 may automatically extract context 82 of the radiology examination. These request items 80, 82 provided with the initiation are considered fixed items 80, because they are already implicitly chosen by the requestor and are therefore not inferred by an AI algorithm of the system 54. The remaining request items 84 (i.e., the remaining fields of a request form 88) not provided with the request initiation 80 are proposed by an AI algorithm 90, based on the selected fixed items 80 and the process context 82. The process context 82 includes all real-time status information and resource assignments of all running process instances in the radiology department that pertain to the radiology examination workflow, as provided by a workflow management system. For example, the process context includes, but is not limited to: predicted, scheduled, or historic start and end times of all activities; assignment of human resources to each activity, e.g. who is currently working on which activity; assignment of rooms, devices, and other resources to each activity; and/or so forth.

The AI algorithm 90 proposes the missing request items based on historic requests and/or other information. The AI algorithm 90 can be implemented using a decision tree model, an artificial neural network (ANN), a support vector machine (SVM), or the like. The AI algorithm 90 determines probabilities for each possible value of the proposed request items. The requestor is then presented with the draft request form 88 that includes the fixed item(s) 80 and proposed request item(s) 84. Because the AI algorithm 90 may not correctly predict values for some request items, the user can select among multiple choices for proposed items, for example using a dropdown list graphical user interface (GUI) dialog as shown by the righthand “down” arrow in the proposed items 84 of FIG. 6. In some embodiments, the items in the dropdown list are sorted according to their probability as determined by the AI algorithm 90. In some embodiments, the user dialog of the proposed items 84 additionally or alternatively enables the requestor to choose to enter a value that was not proposed by the AI algorithm 90.

Whenever the requestor selects a value for any of the proposed request items 84, this item may then be considered a fixed item, and the AI algorithm 90 may optionally be re-run as indicated by the flowback arrow 92 in FIG. 6, considering the recently selected item as an additional fixed item to reevaluate the probabilities for the remaining request items. Once the requestor has selected or entered all required request items (that is, has filled in all fields of the request form), the draft request for assistance 88 is finalized and the draft request form 88 becomes an approved request form 94. The approved request for assistance 94 will then trigger a communication event or the creation of a ticket. For example, the approved request for assistance 94 may be presented on a second person's (e.g., the recipient's) electronic device which is accessible by the second person (e.g. recipient). In some embodiments, the recipient may be a group of persons, e.g. if the request is for transport assistance then the approved request for assistance 94 may be broadcasted over the hospital intercom. In some embodiments, the approved request for assistance 94 may also be transmitted to supervisory personnel such as operational managers.

Optionally, each approved request form 94 and its counterpart (initial) draft request form 88 serve as training data for further training of the AI algorithm 90. In this training data, each proposed request item 84 generated by the AI algorithm 90 in the draft request from 88 which is included unmodified in the approved request form 94 constitutes positive reinforcement in which the output of the AI algorithm 90 is confirmed; whereas, each proposed request item 84 generated by the AI algorithm 90 in the draft request from 88 which is changed by the requestor so that its value in the approved request form 94 is different from that included in the initial draft request form 88 constitutes negative reinforcement in which the output of the AI algorithm 90 is rejected (or at least modified). Using this training data, the AI algorithm 90 can be retrained or update trained, thereby improving the accuracy of the AI algorithm 90 as it is used.

With continuing reference to FIG. 6 and further reference to FIG. 7, as previously noted some embodiments of the process-aware assistance request process performed by the system 54 leverage a workflow management system that maintains information and optionally a predictive model of the radiology examination workflow. FIG. 7 illustrates a GUI representation of a radiology examination workflow timeline 100 which may for example be presented on the display of the electronic devices 16, 32, 38, 42, 44 (see FIG. 1) of those persons participating in the workflow. FIG. 7 shows an example of the radiology examination workflow timeline 100 of the radiology examination workflow for an MRI examination presented to an MRI technologist, which gives an overview of running and upcoming MRI processes as provided by a workflow management system. In this example, the MRI technologist wishes to initiate a request for assistance during the “Executing Cardiac MRI” phase of the MRI examination, for example to ask the on-call radiologist 34 for advice regarding an intended change in an imaging protocol. (Hence, in this example the “requestor” is the MRI technologist, corresponding to the imaging technologist 20 of FIG. 1, and the intended requestee is the on-call radiologist 34 of FIG. 1). To do so, the MRI technologist clicks on a point in the “Executing Cardiac MRI” phase of the MRI examination workflow timeline 100 (as diagrammatically shown in FIG. 7 by a mouse cursor 102 at that point). When clicking on the respective activity, the system of FIG. 6 pre-populates the request items “patient”, “activity type”, and “time”, because the element in the radiology examination workflow timeline 100 is already linked to all of these. Hence, these become fixed items 80 of the draft request for assistance 88. The AI algorithm 90 will then propose the most probable entries for the remaining items 84. For example, from historic communication events, the AI model 90 may have learned that the most common recipient for requests concerning this activity type is the on-call radiologist. Further, the AI model 90 may have learned that, given the context 82 of the request, the most common request type is “protocol modification approval”. Further, the AI model 90 may have learned that, given the context of the request, the most probable urgency level of this request is “very urgent”. Accordingly, the predicted recipient, request type, and urgency level are pre-populated as proposed items 84 in the draft request form 88.

More generally, the request initiation depicted in FIG. 7 entails receiving a selection 102 of a time on the timeline 100 via the requestor's electronic device, and in this case the context 82 of the radiology examination used in the producing of the draft communication request 88 is a context of the radiology examination at the selected time. Even more generally, in some embodiments the radiology examination is an in-progress radiology examination at a time the inquiry is received, and the context 82 of the radiology examination used in the producing of the draft communication request 88 is a current context of the in-progress radiology examination.

It will be noted that in most cases the inquiry at least indicates a personnel role of the requestor in the radiology examination (since the electronic device from which the inquiry initiates is associated to the requestor) and hence the producing of the draft communication request 88 will typically include filling in at least one field of the communication request form (e.g., the identity of the requestor) based on the indicated personnel role of the requestor. More generally, the draft request 88 may include various metadata such as exam name, nature of assistance needed, and/or so forth.

FIG. 7 presents one illustrative example, but it will be appreciated that a representation of the ongoing radiology examination workflow can be similarly used to facilitate initiating requests for assistance between other participants in the radiology examination workflow. As another example, a participant may have a question for the nurse 26 of the patient's ward 22 (see FIG. 1). The user clicks on the patient's name, allowing the assistance request system 54 to pre-populate the patient as fixed request item 80. Other request items are not yet uniquely defined; hence, the AI model 90 fills these in as proposed items 84. For example, the AI model 90 may have learned that the most probable recipient for requests with only the patient name pre-populated at this time of the day is the radiology department receptionist (not shown in FIG. 1), while the second most probable is the nurse 26 on the ward 22. The proposed item is therefore “Receptionist” but the user can instead select “Nurse” from the dropdown list GUI dialog 84. When this is done, the AI model 90 is re-evaluated with the new fixed recipient item as indicated by the flowback arrow 92 of FIG. 6 and thereby adapts the order of proposed items in the request type.

As another example, a user notices that there is a gap in the schedule and would like to know if a patient's appointment can be shifted. The user clicks on the respective time in the timeline of the UI. The communication request is pre-populated with the time as a single fixed item 80. The AI algorithm 90 may have learned from the process context 82 of earlier events, that requests for a time where there is a gap in the schedule are most commonly targeting the receptionist. The receptionist will therefore be prioritized in the proposed selection of recipients.

As yet another example, the user would like to contact a specific person, for example a specific technologist or radiologist. By clicking on a UI element representing or being uniquely associated with this person, a request proposal is generated with the recipient pre-populated as fixed item 80. Considering the complexity of the currently running MRI examination, the AI algorithm 90 may propose that if the recipient is a radiologist, the request type is a question about the currently running complicated exam and even proposes the respective patient and activity type to be selected in the request.

These are merely nonlimiting illustrative examples.

In some embodiments, a request for assistance can be initiated without any initially fixed request item. (That is, the number of fixed items 80 may be zero). In this case, either the user can activate a generic request function via the GUI, or the AI algorithm 90 may operate based only on the context 82 to decide when to propose the initiation of a request based on, for example, timestamps and process context of historically observed request initiation.

In the above examples, the process-aware assistance request system 54 is initiated by receiving a request for assistance from a requestor. However, in other embodiments, the role-specific examination complexity prediction 52 may be used to initiate the assistance request. In such embodiments, the advice and/or assistance provided to personnel assigned to fill the personnel roles of the workflow based on the determined role-specific examination complexity metrics may include predicting an assistance request by a first person assigned to fill a first personnel role of the workflow directed to a second person assigned to fill a second personnel role of the workflow, and thereby invoking the process of FIG. 6 to automatically generate the draft request for assistance 88 that implements the predicted assistance request, and presenting the draft request for assistance 88 on the first person's electronic device. As previously described with reference to FIG. 6, upon receiving approval of the draft request for assistance 88 via the first person's electronic device, the draft request for assistance becomes the approved request for assistance 94 which is then presented on the second person's electronic device which is accessible by the second person.

The invention has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A radiology workflow assistance apparatus comprising:
a processor; and
a non-transitory storage medium storing instructions readable and executable by the processor to:
retrieve information pertaining to an upcoming radiology examination of a patient including at least information identifying an imaging modality to be used in the upcoming radiology examination, information about the patient, and information obtained from a radiology examination order for the upcoming radiology examination;
based on the retrieved information, determine role-specific examination complexity metrics for respective personnel roles of a workflow for performing the upcoming radiology examination; and
provide at least one of advice or assistance to personnel assigned to fill the personnel roles of the workflow based on the determined role-specific examination complexity metrics.

2. The radiology workflow assistance apparatus of claim 1, wherein, to determine the role-specific examination complexity metrics-includes, the instructions are readable and executable by the processor to further:
determine each role-specific examination complexity metric by applying a role-specific predictive model to the retrieved information.

3. The radiology workflow assistance apparatus of claim 2, wherein the role-specific predictive models are artificial intelligence models.

4. The radiology workflow assistance apparatus of claim 2, wherein, to determine the role-specific examination complexity metrics, the instructions are readable and executable by the processor to further:
derive role-related features from the retrieved information for each respective personnel role of the workflow;
wherein each role-specific examination complexity metric is determined by applying the corresponding role-specific predictive model to the role-related features for that personnel role of the workflow.

5. The radiology workflow assistance apparatus of claim 1 wherein, for at least one personnel role of the workflow:
the role-specific examination complexity metric comprises a multidimensional vector in which each dimension of the multidimensional vector contains a value indicative of role-specific complexity of the upcoming radiology examination with respect to a corresponding aspect of the upcoming radiology examination.

6. The radiology workflow assistance apparatus of claim 1, wherein, to provide at least one of advice or assistance, the instructions are readable and executable by the processor to further present information indicative of the role-specific examination complexity metrics on electronic devices accessible by the personnel assigned to fill the personnel roles of the radiology examination workflow.

7. The radiology workflow assistance apparatus of claim 1, wherein, to at least one of provide advice or assistance, the instructions are readable and executable by the processor to further present information indicative of each role-specific examination complexity metric on one or more electronic devices accessible by one or more persons assigned to fill the personnel role of the workflow corresponding to that role-specific examination complexity metric.

8. The radiology workflow assistance apparatus of claim 7, wherein the presenting of each role-specific examination complexity metric includes:
displaying a schedule of radiology examinations including the upcoming radiology examination on the one or more electronic devices accessible by the one or more persons assigned to fill the personnel role of the workflow corresponding to that role-specific examination complexity metric;
wherein the upcoming radiology examination is color coded in the displayed schedule of radiology examinations based on the role-specific examination complexity metric determined for the upcoming radiology examination.

9. The radiology workflow assistance apparatus of claim 1, wherein, to provide at least one of advice or assistance, the instructions are readable and executable by the processor to further:
predict an assistance request by a first person assigned to fill a first personnel role of the workflow directed to a second person assigned to fill a second personnel role of the workflow;
automatically generate a draft request for assistance that implements the predicted assistance request; and
present the draft request for assistance on a first person's electronic device which is accessible by the first person.

10. The radiology workflow assistance apparatus of claim 9, wherein, to provide at least one of advice or assistance, the instructions are readable and executable by the processor to further:
receive approval of the draft request for assistance via the first person's electronic device whereby the draft request for assistance becomes an approved request for assistance; and
present the approved request for assistance on a second person's electronic device which is accessible by the second person.

11. The radiology workflow assistance apparatus of claim 1, wherein, to provide at least one of advice or assistance the instructions are readable and executable by the processor to further:
predict assistance to a first personnel role of the workflow will be provided by a second personnel role of the workflow; and
automatically schedule an electronic call between one or more persons filling the first personnel role of the workflow and one or more persons filling the second personnel role of the workflow.

12. The radiology workflow assistance apparatus of claim 11, wherein the predicting of assistance includes predicting a time at which the assistance to the first personnel role of the workflow is predicted to be provided by the second personnel role of the workflow, and the automatic scheduling includes scheduling the electronic call for the predicted time.

13. The radiology workflow assistance apparatus of claim 11, wherein the first personnel role of the workflow is a local imaging technologist operating a medical imaging device in the workflow and the second personnel role of the workflow is another imaging technologist.

14. The radiology workflow assistance apparatus of claim 11, wherein the first personnel role of the workflow is a local imaging technologist operating a medical imaging device in the workflow and the second personnel role of the workflow is a radiologist scheduled to approve images acquired by the local imaging technologist in the workflow.

15. The radiology workflow assistance apparatus of claim 1, wherein the instructions are readable and executable by the processor to further:
receive an inquiry regarding a radiology examination via a requestor's electronic device operated by a requestor;
producing produce a draft communication request by filling in fields of a communication request form based on the inquiry and a context of the radiology examination; and
provide a user interface on the requestor's electronic device which displays the draft communication request, enables the requestor to edit the draft communication request, and approve the draft communication request whereby the draft communication request becomes an approved communication request; and
send the approved communication request to a recipient identified in the approved communication request.

16. The radiology workflow assistance apparatus of claim 15, wherein the producing of the draft communication request includes:
filling in a first subset of the fields of the communication request form with fixed items determined based on the inquiry and a current context of the radiology examination;
filling in a second subset of the fields of the communication request form with proposed items based on the inquiry and a current context of the radiology examination.

17. The radiology workflow assistance apparatus of claim 16, wherein the filling in of the second subset of the fields of the communication request form with the proposed items includes applying an artificial intelligence model to the inquiry and a current context of the radiology examination.

18. The radiology workflow assistance apparatus of claim 15, wherein the radiology workflow assistance method further includes:
presenting a timeline of a workflow of the radiology examination on the requestor's electronic device;
receiving a selection of a time on the timeline via the requestor's electronic device;
wherein the context of the radiology examination used in the producing of the draft communication request is a context of the radiology examination at the selected time.

19. The radiology workflow assistance apparatus of claim 15, wherein the radiology examination is an in-progress radiology examination at a time the inquiry is received, and the context of the radiology examination used in the producing of the draft communication request is a current context of the in-progress radiology examination.

20. The radiology workflow assistance apparatus of claim 15, wherein the inquiry indicates a personnel role of the requestor in the radiology examination and the producing of the draft communication request includes filling in at least one field of the communication request form based on the indicated personnel role of the requestor.
